(19) United States
(12) Patent Application Publication
(10) Pub. No.: US 2014/0100473 A1
(43) Pub. Date: Apr. 10, 2014

(54) SYSTEMS AND PROCESSES FOR CORTICAL INTEGRITY ASSESSMENT

(71) Applicant: Todd O'Brien, Orono, ME (US)

(72) Inventor: Todd O'Brien, Orono, ME (US)

(21) Appl. No.: 14/046,559

(22) Filed: Oct. 4, 2013

Related U.S. Application Data
(60) Provisional application No. 61/709,632, filed on Oct. 4, 2012.

(51) Int. Cl. A61B 5/00

(52) U.S. Cl. CPC A61B 5/4509; A61B 5/0051
USPC 600/552

(57) ABSTRACT

An enhanced system and process for assessment of cortical integrity is disclosed. The system permit detection of cortical disruption through identification of at least one of frequency shifts and significantly reduced vibration transmission in injured versus non-injured bones. Radiography and concomitant exposure to the same is reduced along with risks and costs of the same.

### RELATED APPLICATIONS

This application claims the full Paris Convention priority to, and benefit of, U.S. Provisional Application Ser. No. 61/709,632, filed Oct. 4, 2012, the contents of which are incorporated by this reference as if fully set forth herein in their entirety.

### BACKGROUND OF THE DISCLOSURE

### Field of Inventions

Vibrational stimuli have long been used in research and clinical settings to assess bone integrity. The most common current example of this in clinical practice is the use of ultrasound to assess bone density. This technique involves the transmission of high frequency sound waves through bone to a receiver which interprets the resulting vibrations. These results are then compared to known standards and a clinical diagnosis can be derived regarding bone density. Such devices have numerous issues, including little to no applicability in the field, difficulty in interpreting, and may foster serious deleterious impacts for want of data to make clean assessments.

Research in biomedical engineering, biomechanics, and orthopedics has produced many studies evaluating bone density, fracture healing, prostheses loosening and joint mechanics through the application of vibration. Despite these advances in our understanding including research-derived prior art on the subject, no widely available, cost-effective instruments have made their way into clinical practice for the evaluation of fractures, prior to the advent of the instant technique. Alternatively, clinicians have developed a simple but non-quantitative technique for fracture identification though the use of a 128 Hz tuning fork and stethoscope. In this method, the examiner places a stethoscope over a boney prominence at the end of a long bone and a vibrating 128 Hz tuning fork at the opposite end of the same bone. The examiner compares the sound of the bone on the presumably injured side to the same bone on the uninjured side. Experienced examiners are able to appreciate differences in the sounds of intact versus fractured bone. This simple test has been advocated for situations where x-rays are not available such as at sporting events or in remote locations (Lipmann, R K. “The Use of Auscultatory Percussion for the Examination of Fractures.” J Bone Joint Surg Am, 1932 Jan. 1;14(1):118-126; Moore, M B. “The Use of a Tuning Fork and Stethoscope to Identify Fractures.” Journal of Athletic Training 2009;44 (3):272-274; Misurya R K, Khare A, Mallick A, Sural A, Vishwakarma G K. “Use of tuning fork in diagnostic auscultation of fractures.” Injury. 1987 Jan. 18 (1):63-4.). Clearly, this is indicative of a need to offer enhancements for consideration.

Another instance of using tuning forks to improve the specificity of fracture identification has been reported by Dissmann and Han in their article on Ottawa ankle rules (Dissmann, P D, Han K H. The tuning fork test—a useful tool for improving specificity in “Ottawa positive” patients after ankle inversion injury. Emerg Med J 2006;23:788-790. doi: 10.1136/emj.2006.035519). Ottawa ankle rules are clinical guidelines that have been developed to help reduce missed foot and ankle fractures. When used properly they have been shown to reduce the need for unnecessary radiographs, thereby reducing radiation exposure to patients and costs to an already overburdened healthcare system. Although, Dissmann and Han use patient pain feedback instead of auscultation in order to improve their ability to rule out fractures, their study highlights the need to reduce unnecessary radiographs, among other desiderata for improved therapies.

### SUMMARY OF THE DISCLOSURE

According to embodiments, there are provided software applications, and related hardware system embodiments, and variations thereon for utilizing frequency shifts to identify fractures. Further, simply elegant methods with conventional tools are shown.

Likewise, systems, methods and applications applying frequency shifts, particularly at a structure's resonant frequency are within the scope of the instant teachings, particularly for identifying what those skilled in the art understand to be Level 1 Damage, or that which is actually present in the structure.

Further envisioned are embodiments utilizing conventional apparatus, for example, the microphone function of a smartphone, to analyze vibrations transmitted through bone has been explored and provides complementary data, as a rudimentary example.

In such cases, the vibration source may be another smartphone or any other device that has a constant vibrational frequency and amplitude output. In the preferred embodiment, the same phone serves as vibrator and microphone. In this case the microphone is attached to cord long enough to allow placement where desired. The phone itself is then situated appropriately in contact with the body while vibrating. Boney prominences serve as ideal locations for placement of the phone and the microphone. The peak vibration frequency and amplitudes are then measured on injured and uninjured sides. The application analyzes the data and compares amplitudes for significant differences. A significantly reduced amplitude on one side indicates a disruption in the cortical integrity of the involved bone. Depending on the clinical context, this would most likely represent a fracture or other abnormality including the loosening of a prosthetic device.

According to embodiments, there is disclosed a system for cortical integrity assessment, which comprises, in combination; at least a means for analyzing vibrational stimuli transmitted through bone, and, further comprising a means for detection of cortical disruption of significantly reduced vibration transmission in injured versus non-injured bones.

According to embodiments, there is disclosed a system for cortical integrity assessment, further comprising at least a software application including data registration and processing accumulated by applying an input and measuring output.

According to embodiments, there is disclosed a system for cortical integrity assessment, which comprises, in combination, at least applying vibration to a subject bone, and then measuring the vibration as modulated by the bone.

According to embodiments, there is disclosed a system for cortical integrity assessment, further comprising analyzing a resultory output signal.

According to embodiments, there is disclosed a system for cortical integrity assessment, whereby fractures are identified by at least one method selected from the group of frequency response assessment and reduction in amplitude measurement.

According to embodiments, there is disclosed a system for cortical integrity assessment, the frequency response being measured by application of a wide frequency spectrum and calculation of impulse response by deconvolution of the input signal and an output signal of the system.

The present invention offers for consideration techniques for the rapid, inexpensive and quantitative assessment of cortical bone integrity in non-clinical settings that has not been previously contemplated in the prior art. This technique can also be used to screen patients within the healthcare system potentially reducing the performance of unnecessary radiographs.

Briefly stated, an enhanced system and process for assessment of cortical integrity is disclosed. The system permit detection of cortical disruption through identification of at least one of frequency shifts and significantly reduced vibration transmission in injured versus non-injured bones. Radiography and concomitant exposure to the same is reduced along with risks and costs of the same.

Testing on intact and fractured bone using the two phone technique and an acoustical engineering application has been performed (FIGS. 3, 4). Results confirm a 30% reduction in the magnitude of the peak frequency vibrations transmitted across the fracture site compared to intact bone.

### BRIEF DESCRIPTION OF THE DRAWINGS

FIG. 1 shows use of frequency shifts to identify ostensive fractures, according to embodiments of the present invention;

FIG. 2 graphically depicts frequency response in bone, according to embodiments of the present invention;

FIG. 3 graphically depicts frequency response in fractured bone, according to embodiments of the present invention;

FIG. 4 is a schematic flow chart according to embodiments of the present invention;

FIG. 5 is a depiction of the one-smartphone method for cortical integrity assessment according to embodiments of the present invention; and,

FIG. 6 is a depiction of the two-smartphone method for cortical integrity assessment according to embodiments of the present invention.

### DETAILED DESCRIPTION OF THE DISCLOSURE

The present inventor has discovered that the important clinical goal and under-resourced aspect of treatment which is key to fracture identification in bones is essential to management and treatment of such insult, injury, and trauma to bones. It is an object of the present invention to provide a system and process for assessment of cortical integrity, utilizing software applications to analyze vibrational stimuli transmitted through bone. This assessment may be performed several ways which advance progress in science and the useful arts.

It is another object of the present invention to enable detection of cortical disruption through identification of significantly reduced vibration transmission in injured versus non-injured bones.

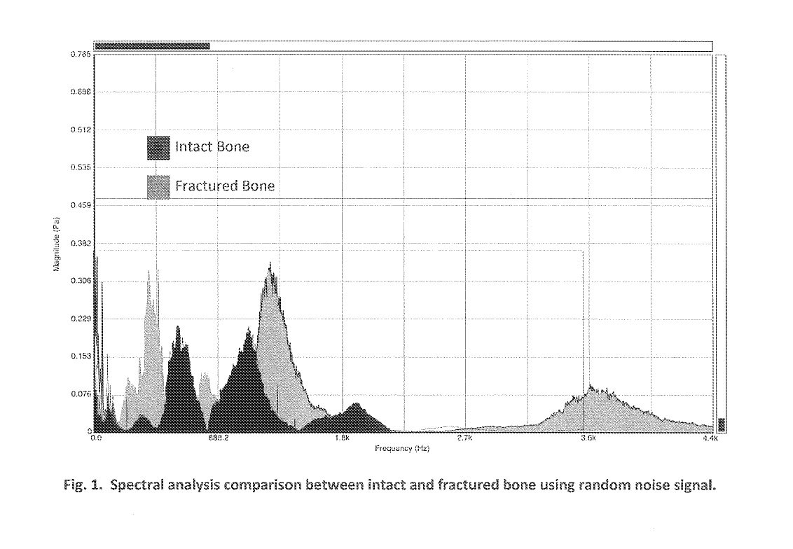

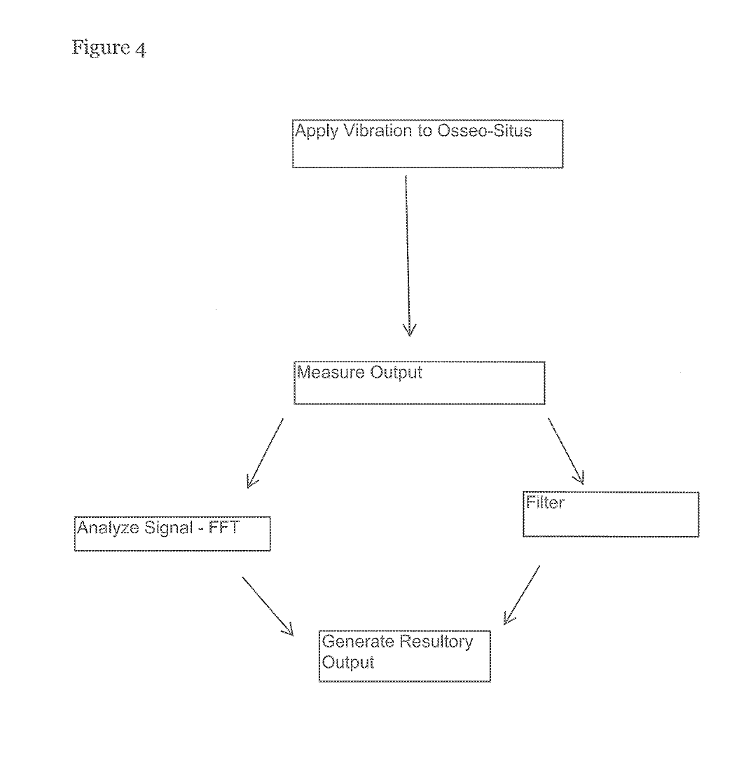

Referring now to FIG. 1, an exemplary embodiment uses frequency shift to identify ostensive fractured bones. This figure shows a spectral analysis comparison between intact and fractural bone, using random noise signal. Magnitude is arrayed versus frequency, showing data computable by software (see, FIG. 4).

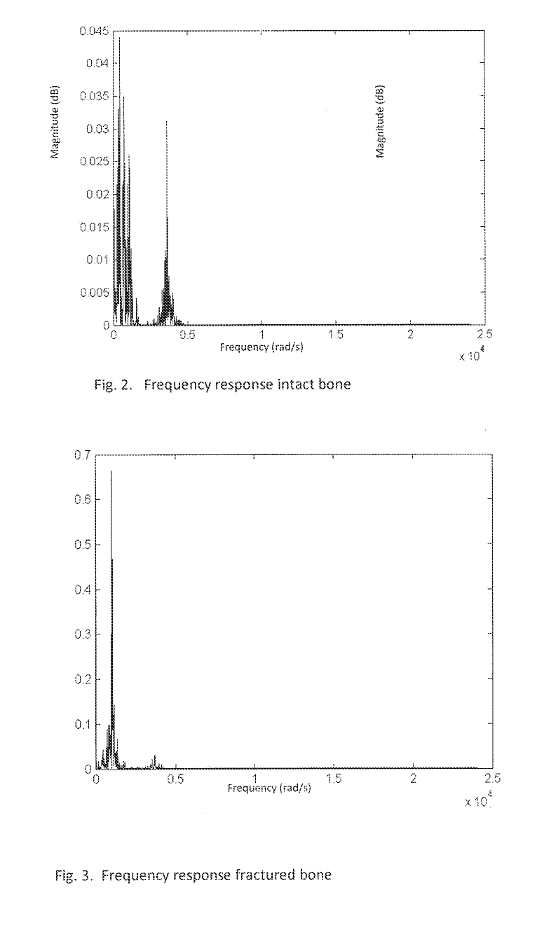

Referring now to FIGS. 2 and 3, the frequency response function method of fracture identification is shown. Frequency response is the quantitative measure of the output spectrum of a system or device in response to a stimulus. It is a measure of magnitude and phase of the output as a function of frequency, in comparison to the input. Estimating the frequency response for a physical system generally involves exciting the system with an input signal, measuring both input and output time histories, and comparing the two through a process such as the Fast Fourier Transform (FFT). Accordingly, the frequency response of a system can be measured by applying a wide frequency spectrum (for example, digitally generated maximum length sequence noise, or an analog filtered white noise equivalent, like pink noise) and calculating the impulse response by deconvolution of this input signal and the output signal of the system. This technique can be used to assess the integrity of bone and thereby identify fracture as demonstrated in FIGS. 2 and 3.

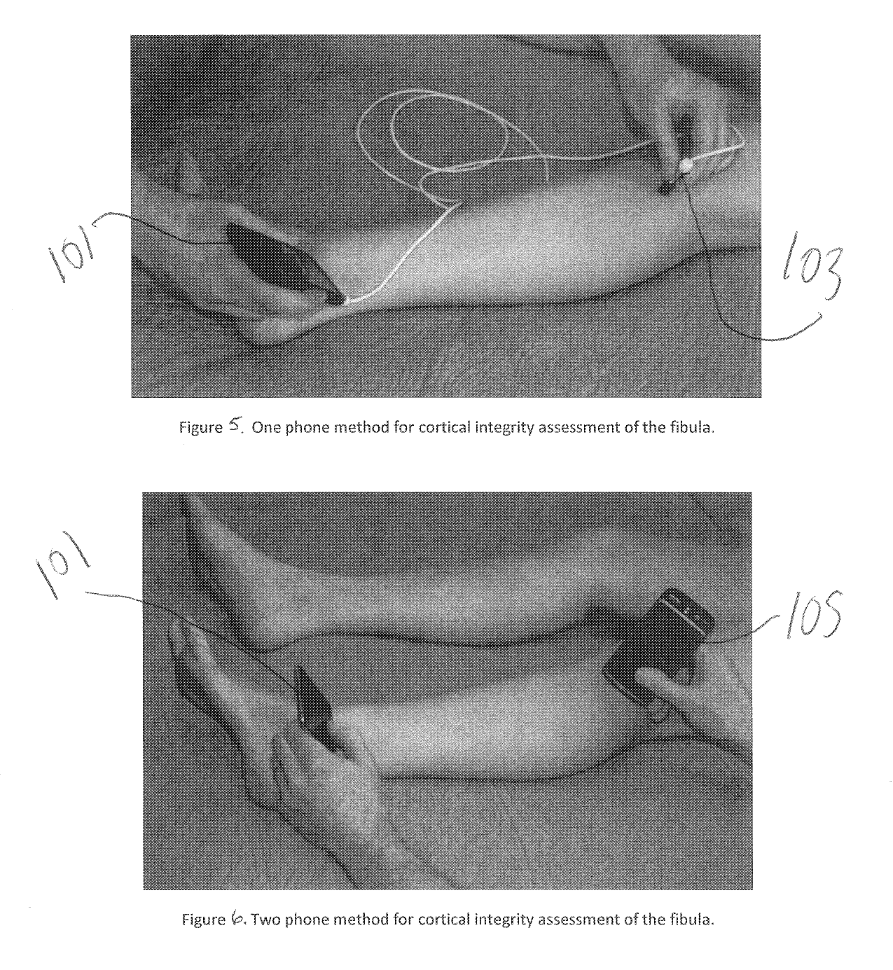

Referring now to FIGS. 5 and 6, conventional smartphone 101 and microphone 103 are arrayed per instructions on screen of conventional smartphone 101. The two bone locations (uninjured and ostensively injured) are tested, and peak frequency amplitude readings displayed on screen of conventional smartphone 101. For the two-smartphone method, conventional smartphone 101 runs the application and conventional smartphone 105 houses a vibration-measurement application.

A utilization protocol for the single phone embodiment is as follows: 1.) the application is started with selection of the one phone protocol; 2.) the appropriate bone is selected for analysis; 3.) instructions for placement of the phone and microphone are displayed; 4.) the uninjured side is tested followed by the injured side; 5.) the application compares peak frequency amplitudes of each side; 6.) results are displayed indicating if a significant difference exists between the two sides.

A utilization protocol for the two phone embodiment is as follows: 1.) the application is started with selection of the two phone protocol; 2.) the appropriate bone is selected for analysis. Instructions for placement of the phones are displayed; 3.) the phone running the application serves as the microphone, the other is the vibrator; 4.) a vibrator application will need to be installed on this phone—alternatively, any device with a constant vibrational output can be used; 6.) the phones are placed on appropriate anatomic landmarks and testing of the uninjured and injured side is performed; 7.) the top of each phone is held vertically over these locations; 8.) the application compares peak frequency amplitudes of each side; 10.) results are displayed indicating if a significant difference exists between the two sides.

While the method and apparatus have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all references listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant.

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A system for cortical integrity assessment, which comprises, in combination:
at least a means for analyzing vibrational stimuli transmitted through bone; and,
further comprising a means for detection of cortical disruption of significantly reduced vibration transmission in injured versus non-injured bones.

2. The system of claim 1, further comprising at least a software application including data registration and processing accumulated by applying an input and measuring output, to generate resultory plotted results.

3. The system of claim 2, which comprises, in combination, at least applying vibration to a subject bone, and then measuring the vibration as modulated by the bone.

4. The system of claim 1, further comprising analyzing a resultory output signal.

5. The system of claim 4, whereby fractures are identified by at least one method selected from the group of frequency response assessment and reduction in amplitude measurement.

6. The system of claim 5, the frequency response being measured by application of a wide frequency spectrum and calculation of impulse response by deconvolution of the input signal and an output signal of the system.

7. The system of claim 4, further comprising at least a conventional smartphone effective for running a subject application.

8. The system of claim 7, further comprising at least two conventional smartphones, each effective for running a subject application.

9. The system of claim 8, wherein the application further comprises output and display means for showing results of processed data.

10. A process for determining cortical integrity via frequency shifts measured from east two bone density measurements.

11. The process of claim 10, further comprising software.

12. The process of claim 11, further comprising smart electronics.

13. The process of claim 12, further comprising a vibrator and a microphone.

14. The process of claim 13, further comprising a database.

15. An application for determining cortical integrity, which comprises, in combination:
at least one mechanism selected from the group of frequency shifts and changes in vibration transmission for assessing injured versus non-injured bones;
wherein data sets from at least an ostensively injured situs and control are compared.
